(19) United States
(12) Patent Application Publication
(10) Pub. No.: US 2024/0423827 A1
(43) Pub. Date: Dec. 26, 2024

(54) BITE GUARD WITH EMBEDDED PRESSURE SENSORS

(71) Applicant: INTERNATIONAL BUSINESS MACHINES CORPORATION, ARMONK, NY (US)

(72) Inventors: Jeremy R. Fox, Georgetown, TX (US); Martin G. Keen, Cary, NC (US); Alexander Reznicek, Troy, NY (US); Bahman Hekmatshoartabari, White Plains, NY (US)

(21) Appl. No.: 18/337,810

(22) Filed: Jun. 20, 2023

(51) Int. Cl. A61F 5/56

(52) U.S. Cl. CPC A61F 5/566; A61F 2005/563

(57) ABSTRACT

A bite guard configured to conform to teeth of a patient, and an array of sensor assemblies encapsulated in the bite guard, where the array of sensor assemblies is distributed throughout the bite guard, and where each sensor assembly includes a piezo pressure sensor.

### BACKGROUND

Aspects of the present disclosure relate to a bite guard with embedded pressure sensors.

Bite guards, also known as mouth guards or occlusal splints, are dental devices designed to protect the teeth, jaws, and surrounding structures from damage caused by teeth grinding (bruxism), clenching, or other parafunctional habits. These habits can occur during sleep or even during waking hours due to stress or misalignment of the teeth. Bite guards act as a cushioning barrier, absorbing the forces exerted during grinding or clenching, and distributing them evenly to minimize the impact on the teeth and jaw joints. They are typically made of a durable, flexible material that is custom fitted to the individual's mouth for maximum comfort and effectiveness. Bite guards are commonly used in the fields of dentistry and sports dentistry to prevent tooth wear, fractures, TMJ disorders, and muscle tension, promoting oral health and providing relief from related symptoms.

### BRIEF SUMMARY

The present disclosure provides a device, computer program product, and system related to a bite guard with embedded pressure sensors. In some embodiments, the device includes a bite guard configured to conform to teeth of a patient, and an array of sensor assemblies encapsulated in the bite guard, where the array of sensor assemblies is distributed throughout the bite guard, and where each sensor assembly includes at least one piezo pressure sensor.

Some embodiments of the present disclosure can also be illustrated by a computer program product comprising a computer readable storage medium having program instructions embodied therewith, the program instructions executable by a processor to cause the processors to perform a method, the method comprising detecting, by an array of piezo sensor assemblies in a bite guard, pressure measurements applied by a patient to the bite guard, and transmitting, from the array of piezo sensor assemblies in the bite guard to the processor, the pressure measurements. The computer program product may further comprise additional program instructions stored on the computer readable storage medium and configured to cause the processor to perform the method further comprising: detecting, by the array of piezo sensor assemblies, location data for the array of piezo sensor assemblies; transmitting, from the array of piezo sensor assemblies in the bite guard to the processor, the location data, mapping the array of piezo sensor assemblies with an individual location and orientation of the sensors in the bite guard, and generating, based on the mapping of the location and the pressure measurements, a time-dependent pressure map.

Some embodiments of the present disclosure can also be illustrated by a system comprising a processor and a memory in communication with the processor, the memory containing program instructions that, when executed by the processor, are configured to cause the processor to perform a method, the method comprising detecting, by an array of piezo sensor assemblies in a bite guard, pressure measurements applied by a patient to the bite guard, and transmitting, from the array of piezo sensor assemblies in the bite guard to the processor, the pressure measurements. In some embodiments of the present disclosure, the memory stores further program instructions, and where the processor is configured to execute the further program instructions to perform the processes further comprising: detecting, by the array of piezo sensor assemblies, location data for the array of piezo sensor assemblies, transmitting, from the array of piezo sensor assemblies in the bite guard to the processor, the location data, mapping the location of the array of piezo sensor assemblies in the bite guard, and generating, based on the mapping of the location and the pressure measurements, a time-dependent pressure map.

### BRIEF DESCRIPTION OF THE DRAWINGS

FIG. 1 illustrates an example computing environment, according to various embodiments of the present invention.

FIG. 2 depicts a computer system, according to various embodiments of the present invention.

FIG. 3A depicts bite guard with embedded pressure sensors, according to various embodiments of the present invention.

FIG. 3B depicts a simplified cross section of the pressure sensors, according to various embodiments of the present invention.

FIG. 4 depicts a method of fabricating and a method of operating a bite guard detection device, according to various embodiments of the present invention.

FIG. 5 depicts an example bite guard system, according to various embodiments of the present invention.

FIG. 6 depicts an example method of fabricating a bite guard, measuring pressure and orientation data from embedded sensor assemblies, and analyzing the data to form a pressure map, according to various embodiments of the present invention.

### DETAILED DESCRIPTION

Aspects of the present disclosure relate to a bite guard with embedded pressure sensor assemblies that include a piezo pressure sensor, a MEMS (Micro-Electro-Mechanical Systems) position sensor, and a communication chip. While the present disclosure is not necessarily limited to such applications, various aspects of the disclosure may be appreciated through a discussion of various examples using this context.

Teeth grinding problems can be difficult to recognize, as the effects are often only visible in loss of teeth material. This makes it hard for medical professionals to identify the root cause of teeth grinding and provide timely treatment.

Insights and data that can then be used to accurately diagnose and treat teeth grinding issues is needed.

The present disclosure provides medical professionals with an innovative way to accurately measure and monitor the pressure on a person's teeth and can help them diagnose and treat problems much earlier than before to help address the problem.

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

Computing environment 100 contains an example of an environment for the execution of at least some of the computer code involved in performing the inventive methods, such as fabricating a bite guard and measuring the pressure asserted on the bite guard by a patient's teeth with code 200 In addition to code 200, computing environment 100 includes, for example, computer 101, wide area network (WAN) 102, end user device (EUD) 103, remote server 104, public cloud 105, and private cloud 106. In this embodiment, computer 101 includes processor set 110 (including processing circuitry 120 and cache 121), communication fabric 111, volatile memory 112, persistent storage 113 (including operating system 122 and code 200, as identified above), peripheral device set 114 (including user interface (UI), device set 123, storage 124, and Internet of Things (IoT) sensor set 125), and network module 115. Remote server 104 includes remote database 130. Public cloud 105 includes gateway 140, cloud orchestration module 141, host physical machine set 142, virtual machine set 143, and container set 144.

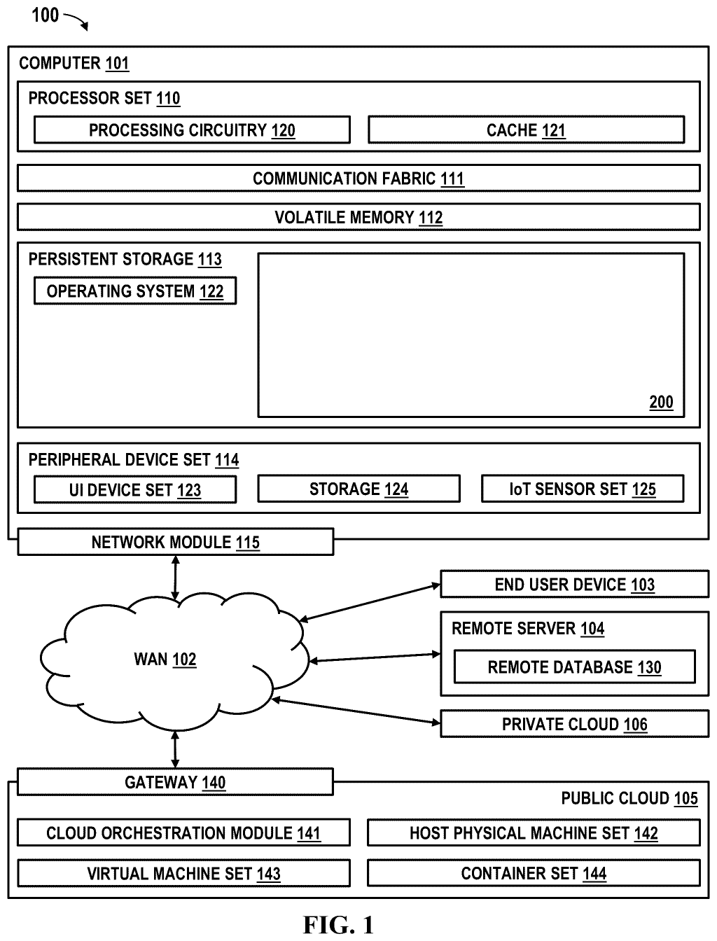

COMPUTER 101 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 130. As is well understood in the art of computer technology, and depending upon the technology, performance of a computer-implemented method may be distributed among multiple computers and/or between multiple locations. On the other hand, in this presentation of computing environment 100, detailed discussion is focused on a single computer, specifically computer 101, to keep the presentation as simple as possible. Computer 101 may be located in a cloud, even though it is not shown in a cloud in FIG. 1. On the other hand, computer 101 is not required to be in a cloud except to any extent as may be affirmatively indicated.

PROCESSOR SET 110 includes one, or more, computer processors of any type now known or to be developed in the future. Processing circuitry 120 may be distributed over multiple packages, for example, multiple, coordinated integrated circuit chips. Processing circuitry 120 may implement multiple processor threads and/or multiple processor cores. Cache 121 is memory that is located in the processor chip package(s) and is typically used for data or code that should be available for rapid access by the threads or cores running on processor set 110. Cache memories are typically organized into multiple levels depending upon relative proximity to the processing circuitry. Alternatively, some, or all, of the cache for the processor set may be located “off chip.” In some computing environments, processor set 110 may be designed for working with qubits and performing quantum computing.

Computer readable program instructions are typically loaded onto computer 101 to cause a series of operational steps to be performed by processor set 110 of computer 101 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 121 and the other storage media discussed below. The program instructions, and associated data, are accessed by processor set 110 to control and direct performance of the inventive methods. In computing environment 100, at least some of the instructions for performing the inventive methods may be stored in code 200 in persistent storage 113.

COMMUNICATION FABRIC 111 is the signal conduction paths that allow the various components of computer 101 to communicate with each other. Typically, this fabric is made of switches and electrically conductive paths, such as the switches and electrically conductive paths that make up busses, bridges, physical input/output ports and the like. Other types of signal communication paths may be used, such as fiber optic communication paths and/or wireless communication paths.

VOLATILE MEMORY 112 is any type of volatile memory now known or to be developed in the future. Examples include dynamic type random access memory (RAM) or static type RAM. Typically, the volatile memory is characterized by random access, but this is not required unless affirmatively indicated. In computer 101, the volatile memory 112 is located in a single package and is internal to computer 101, but, alternatively or additionally, the volatile memory may be distributed over multiple packages and/or located externally with respect to computer 101.

PERSISTENT STORAGE 113 is any form of non-volatile storage for computers that is now known or to be developed in the future. The non-volatility of this storage means that the stored data is maintained regardless of whether power is being supplied to computer 101 and/or directly to persistent storage 113. Persistent storage 113 may be a read only memory (ROM), but typically at least a portion of the persistent storage allows writing of data, deletion of data and re-writing of data. Some familiar forms of persistent storage include magnetic disks and solid state storage devices. Operating system 122 may take several forms, such as various known proprietary operating systems or open source Portable Operating System Interface type operating systems that employ a kernel. The code included in code 200 typically includes at least some of the computer code involved in performing the inventive methods.

PERIPHERAL DEVICE SET 114 includes the set of peripheral devices of computer 101. Data communication connections between the peripheral devices and the other components of computer 101 may be implemented in various ways, such as Bluetooth connections, Near-Field Communication (NFC) connections, connections made by cables (such as universal serial bus (USB) type cables), insertion type connections (for example, secure digital (SD) card), connections made though local area communication networks and even connections made through wide area networks such as the internet. In various embodiments, UI device set 123 may include components such as a display screen, speaker, microphone, wearable devices (such as goggles and smart watches), keyboard, mouse, printer, touchpad, game controllers, and haptic devices. Storage 124 is external storage, such as an external hard drive, or insertable storage, such as an SD card. Storage 124 may be persistent and/or volatile. In some embodiments, storage 124 may take the form of a quantum computing storage device for storing data in the form of qubits. In embodiments where computer 101 is required to have a large amount of storage (for example, where computer 101 locally stores and manages a large database) then this storage may be provided by peripheral storage devices designed for storing very large amounts of data, such as a storage area network (SAN) that is shared by multiple, geographically distributed computers. IoT sensor set 125 is made up of sensors that can be used in Internet of Things applications. For example, one sensor may be a thermometer and another sensor may be a motion detector.

NETWORK MODULE 115 is the collection of computer software, hardware, and firmware that allows computer 101 to communicate with other computers through WAN 102. Network module 115 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 115 are performed on the same physical hardware device. In other embodiments (for example, embodiments that utilize software-defined networking (SDN)), the control functions and the forwarding functions of network module 115 are performed on physically separate devices, such that the control functions manage several different network hardware devices. Computer readable program instructions for performing the inventive methods can typically be downloaded to computer 101 from an external computer or external storage device through a network adapter card or network interface included in network module 115.

WAN 102 is any wide area network (for example, the internet) capable of communicating computer data over non-local distances by any technology for communicating computer data, now known or to be developed in the future. In some embodiments, the WAN may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

END USER DEVICE (EUD) 103 is any computer system that is used and controlled by an end user (for example, a customer of an enterprise that operates computer 101), and may take any of the forms discussed above in connection with computer 101. EUD 103 typically receives helpful and useful data from the operations of computer 101. For example, in a hypothetical case where computer 101 is designed to provide a recommendation to an end user, this recommendation would typically be communicated from network module 115 of computer 101 through WAN 102 to EUD 103. In this way, EUD 103 can display, or otherwise present, the recommendation to an end user. In some embodiments, EUD 103 may be a client device, such as thin client, heavy client, mainframe computer, desktop computer and so on.

REMOTE SERVER 104 is any computer system that serves at least some data and/or functionality to computer 101. Remote server 104 may be controlled and used by the same entity that operates computer 101. Remote server 104 represents the machine(s) that collect and store helpful and useful data for use by other computers, such as computer 101. For example, in a hypothetical case where computer 101 is designed and programmed to provide a recommendation based on historical data, then this historical data may be provided to computer 101 from remote database 130 of remote server 104.

PUBLIC CLOUD 105 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 105 is performed by the computer hardware and/or software of cloud orchestration module 141. The computing resources provided by public cloud 105 are typically implemented by virtual computing environments that run on various computers making up the computers of host physical machine set 142, which is the universe of physical computers in and/or available to public cloud 105. The virtual computing environments (VCEs) typically take the form of virtual machines from virtual machine set 143 and/or containers from container set 144. It is understood that these VCEs may be stored as images and may be transferred among and between the various physical machine hosts, either as images or after instantiation of the VCE. Cloud orchestration module 141 manages the transfer and storage of images, deploys new instantiations of VCEs and manages active instantiations of VCE deployments. Gateway 140 is the collection of computer software, hardware, and firmware that allows public cloud 105 to communicate through WAN 102.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

PRIVATE CLOUD 106 is similar to public cloud 105, except that the computing resources are only available for use by a single enterprise. While private cloud 106 is depicted as being in communication with WAN 102, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 105 and private cloud 106 are both part of a larger hybrid cloud.

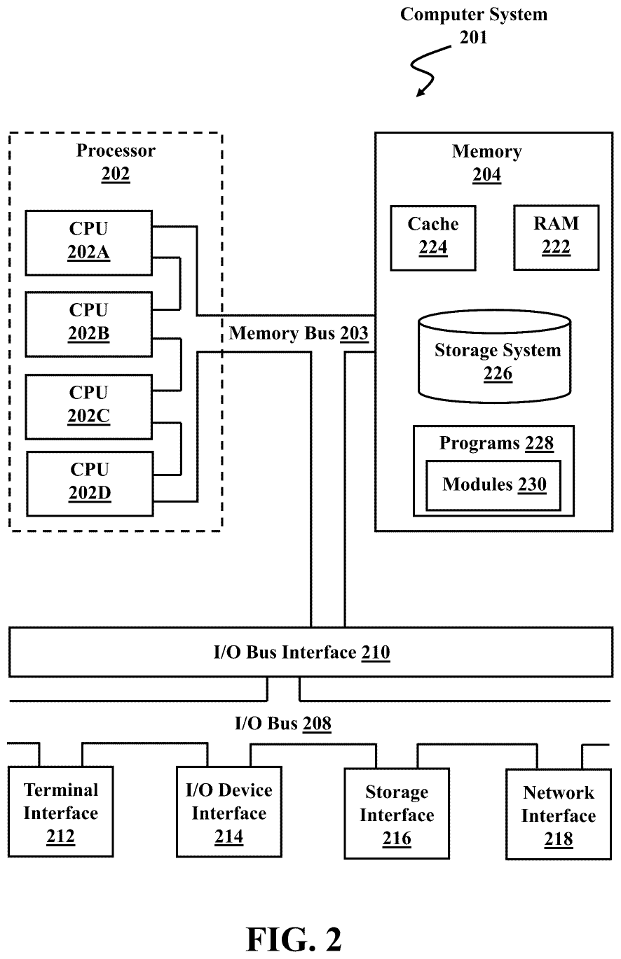

FIG. 2, illustrated is a high-level block diagram of an example computer system 201 that may be used in implementing one or more of the methods, tools, and modules, and any related functions, described herein (e.g., using one or more processor circuits or computer processors of the computer), in accordance with embodiments of the present disclosure. In some embodiments, the major components of the computer system 201 may comprise one or more CPUs 202, a memory subsystem 204, a terminal interface 212, a storage interface 216, an I/O (Input/Output) device interface 214, and a network interface 218, all of which may be communicatively coupled, directly or indirectly, for inter-component communication via a memory bus 203, an I/O bus 208, and an I/O bus interface unit 210.

The computer system 201 may contain one or more general-purpose programmable central processing units (CPUs) 202A, 202B, 202C, and 202D, herein generically referred to as the CPU 202. In some embodiments, the computer system 201 may contain multiple processors typical of a relatively large system; however, in other embodiments the computer system 201 may alternatively be a single CPU system. Each CPU 202 may execute instructions stored in the memory subsystem 204 and may include one or more levels of on-board cache.

System memory 204 may include computer system readable media in the form of volatile memory, such as random access memory (RAM) 222 or cache memory 224. Computer system 201 may further include other removable/non-removable, volatile/non-volatile computer system storage media. By way of example only, storage system 226 can be provided for reading from and writing to a non-removable, non-volatile magnetic media, such as a “hard drive.” Although not shown, a magnetic disk drive for reading from and writing to a removable, non-volatile magnetic disk (e.g., a “floppy disk”), or an optical disk drive for reading from or writing to a removable, non-volatile optical disc such as a CD-ROM, DVD-ROM or other optical media can be provided. In addition, memory 204 can include flash memory, e.g., a flash memory stick drive or a flash drive. Memory devices can be connected to memory bus 203 by one or more data media interfaces. The memory 204 may include at least one program product having a set (e.g., at least one) of program modules that are configured to carry out the functions of various embodiments.

One or more programs/utilities 228, each having at least one set of program modules 230 may be stored in memory 204. The programs/utilities 228 may include a hypervisor (also referred to as a virtual machine monitor), one or more operating systems, one or more application programs, other program modules, and program data. Each of the operating systems, one or more application programs, other program modules, and program data or some combination thereof, may include an implementation of a networking environment. Programs 228 and/or program modules 230 generally perform the functions or methodologies of various embodiments.

Although the memory bus 203 is shown in FIG. 2 as a single bus structure providing a direct communication path among the CPUs 202, the memory subsystem 204, and the I/O bus interface 210, the memory bus 203 may, in some embodiments, include multiple different buses or communication paths, which may be arranged in any of various forms, such as point-to-point links in hierarchical, star or web configurations, multiple hierarchical buses, parallel and redundant paths, or any other appropriate type of configuration. Furthermore, while the I/O bus interface 210 and the I/O bus 208 are shown as single respective units, the computer system 201 may, in some embodiments, contain multiple I/O bus interface units 210, multiple I/O buses 208, or both. Further, while multiple I/O interface units are shown, which separate the I/O bus 208 from various communications paths running to the various I/O devices, in other embodiments some or all of the I/O devices may be connected directly to one or more system I/O buses.

In some embodiments, the computer system 201 may be a multi-user mainframe computer system, a single-user system, or a server computer or similar device that has little or no direct user interface, but receives requests from other computer systems (clients). Further, in some embodiments, the computer system 201 may be implemented as a desktop computer, portable computer, laptop or notebook computer, tablet computer, pocket computer, telephone, smartphone, network switches or routers, or any other appropriate type of electronic device.

It is noted that FIG. 2 is intended to depict the representative major components of an exemplary computer system 201. In some embodiments, however, individual components may have greater or lesser complexity than as represented in FIG. 2, components other than or in addition to those shown in FIG. 2 may be present, and the number, type, and configuration of such components may vary.

In some embodiments, 3D printing technology has enabled the creation of personalized medical devices that can offer precise measurements and enable medical professionals to diagnose and treat patients with more precision. The present disclosure presents a method for using 3D printing technology to create bite guards with embedded pressure sensors that can map the pressure being applied by a person's teeth. This technology may help a medical professional diagnose and treat teeth-grinding problems more effectively, as the pressure patterns may now be mapped using the bite guard.

In some embodiments, the bite guards are made from biocompatible, soft filaments that allow the pressure from the teeth to be accurately transferred to the embedded piezo sensor assembly. In some embodiments, the pressure data from the sensors in the bite guard is received by a handheld device, which then creates a time-dependent pressure map of the person's teeth. This data can also be sent to a server/cloud to be analyzed or shared with a healthcare provider.

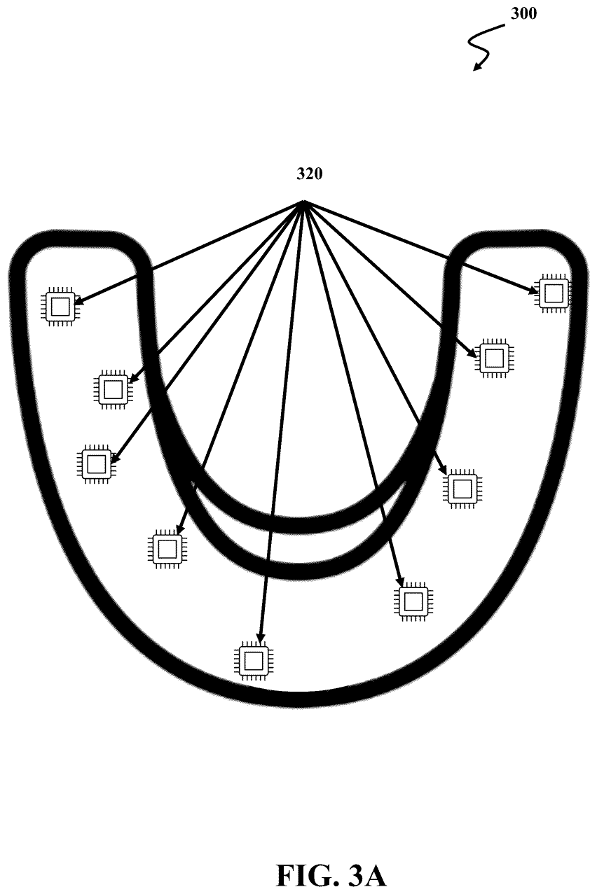

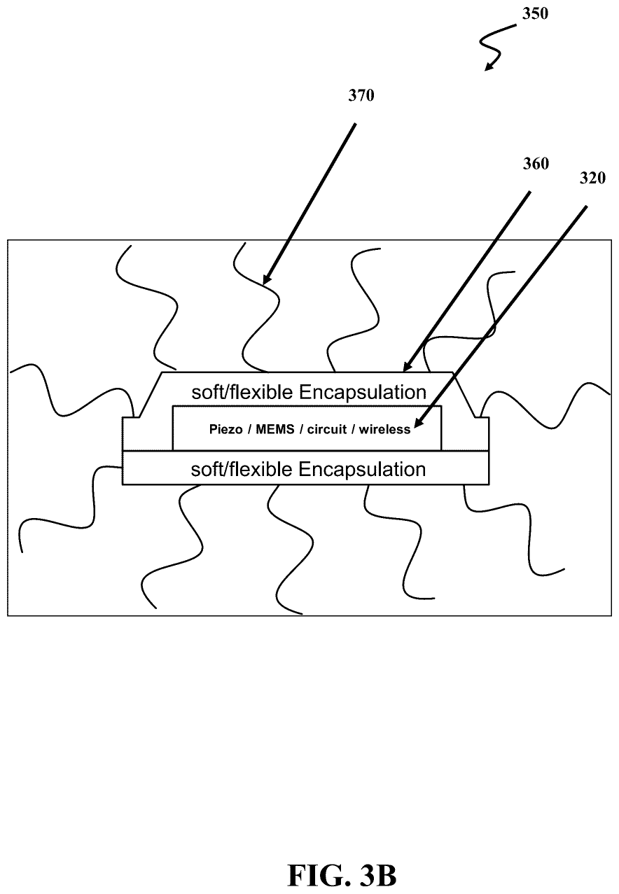

FIG. 3A depicts bite guard 300 (for example, a 3D printed bite guard) with embedded piezo sensor assemblies 320. In some embodiments, bite guard 300 provides an innovative way to accurately measure and monitor the pressure on a person's teeth. This technology can help diagnose and treat teeth grinding problems much more effectively than using traditional methods. In some embodiments, the bite guard itself may be patient-specific and disposable, making it a cost-effective and efficient solution.

FIG. 3B depicts a simplified cross section 350 of one of the piezo sensor assemblies 320 with a soft/flexible encapsulation 360. In some embodiments, the piezo sensor assemblies 320 are randomly embedded piezo sensor assemblies that include piezo pressure sensors and 3D MEMS (Micro-Electro-Mechanical Systems) position sensors, so the orientation of the randomly positioned piezo sensor may be determined. The orientation of the pressure sensors allows for accurate calculation of the force vectors caused by teeth grinding, thereby enabling the capability to extract a full frame pressure map of forces (visualized as lines 370). In some embodiments, each piezo sensor assembly 320 is uniquely identifiable to record its position in the bite guard. After fabrication of the bite guard with the randomly embedded piezo sensor assembly 320, the bite guard is scanned, and a sensor location map is created. Also, a sensor orientation map can be created.

In some instances, a piezo pressure sensor, also known as a piezoresistive pressure sensor, is a device that measures pressure by utilizing the piezoresistive effect. The sensor consists of a thin diaphragm or membrane made of a piezoresistive material, for example silicon, which changes its electrical resistance in response to applied pressure.

In some embodiments, bite guard 300 is composed of a soft matrix material that forms soft/flexible encapsulation 360 (e.g., a 3D printing filament). The soft matrix material allows the transfer of grinding pressure onto the embedded piezo sensor assembly without buffering and distorting of the pressure map.

In some embodiments, each bite guard will have its own unique randomly embedded piezo sensor assembly distribution. The pressure map can then be calculated after receiving individual sensor pressure readings, sensor locations, and sensor orientations from the randomly embedded piezo sensor assembly distribution.

In some embodiments, during teeth grinding a person bites down onto the relatively soft bite guards (e.g., bite guard 300) and triggers the piezo pressure sensors (e.g., piezo sensor assemblies 320). The pressure measured on each sensor depends on the orientation of the sensor relative to the surface of one or more teeth. For example, some piezo pressure sensors may be parallel to the teeth surface, some piezo sensor may be perpendicular to the teeth surface, and other sensors may be at an angle in between parallel and perpendicular to the teeth surface. Thus, since many sensors are embedded (10-100 per cubic centimeter) many orientations will be covered.

In some embodiments, since the orientation is known from 3D MEMS, it is possible to calculate the force vectors x, y, and z, especially when considering neighboring sensors. In some embodiments, the overall pressure map needs to make sense, and the force interactions of neighboring piezo sensors may be used to self-verify the pressure measurements and the resulting maps.

In some embodiments, the pressure map can be used by a medical professional to address certain teeth grinding problems before significant teeth damage.

In some embodiments, the batteries of each sensor assembly can be recharged inductively before or after they are depleted.

In some embodiments, a data receiving apparatus such as a handheld device is used for receiving the pressure data from each of embedded piezo sensor assembly chips at a given time. In some embodiments, the location information and each sensor's pressure data allow the creation of time dependent teeth pressure maps.

In some embodiments, each sensor assembly may include one or more piezo pressure sensors, one or more 3D orientation MEMS chip sets, and/or one or more circuit component for sending a wireless signal to the receiving apparatus. In some instance, the sensor assembly may include a circuit component for sending data used to generate a 3D force vector map.

In some instances, piezo pressure sensors are devices that can measure and detect changes in pressure. They are often used in various applications such as automotive systems, medical devices, and industrial equipment. These sensors can provide accurate pressure readings and are typically compact and durable. Multiple piezo pressure sensors can be combined in a device to monitor pressure levels in different areas or to obtain a more comprehensive understanding of the pressure distribution in a system.

In some instances, 3D orientation MEMS (Micro-Electro-Mechanical Systems) position sensors or MEMS chip sets include gyroscopes, accelerometers, and magnetometers that work together to measure the orientation and movement of an object in three-dimensional space. These chip sets can provide precise data about the object's rotation, tilt, and acceleration. By integrating one or more 3D orientation MEMS chip sets into a device, such as a motion-sensing controller or a navigation system, it becomes possible to accurately track and interpret the object's movements and or position in real-time.

In some instances, circuit components for sending a wireless signal, such as Wi-Fi or Bluetooth® modules, enable wireless communication between devices. In some instances, these components transmit data over radio waves, allowing devices to exchange information without the need for physical connections. By incorporating one or more wireless communication components into a device, it can be made wirelessly accessible, enabling data transfer, remote control, or integration with other wireless-enabled systems. This technology is commonly used in smart home devices, IoT applications, and mobile devices.

In some embodiments, a piezo pressure sensor, 3D orientation MEMS chip set, and/or a circuit component for sending a wireless signal may be combined into a single device. For example, a device could use multiple piezo pressure sensors to monitor pressure distribution in a system, while a 3D orientation MEMS chip set could provide precise orientation data. The device may then utilize a circuit component for sending a wireless signal to transmit the collected data to a mobile device containing a monitoring system or a user interface.

In some embodiments, the signals received from the multitude of sensors may be processed or analyzed locally by the receiving apparatus or transmitted to a server/cloud (and/or shared with a healthcare provider).

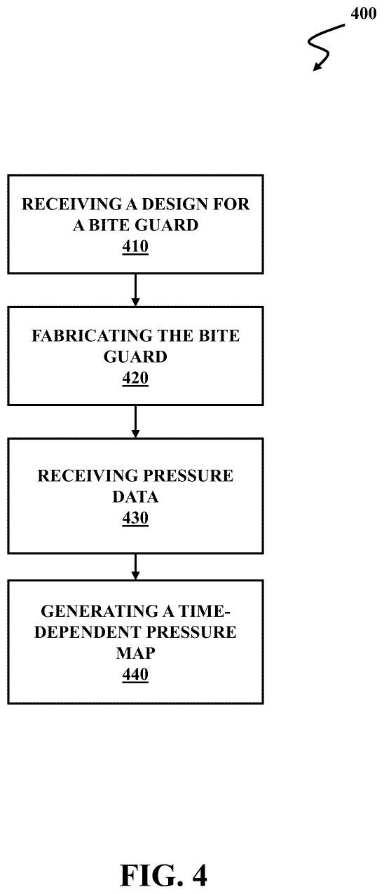

FIG. 4 depicts a method 400 of fabricating a bite guard and later receiving data from a bite guard detection device. In some embodiments, operations in method 400 may be enacted by code 200.

Method 400 begins with operation 410 of receiving a design for a bite guard. In some instances, operation 410 may obtain a digital scan or impression of a patient's teeth and other parts of the mouth. A digital scan or impression of the user's teeth and gums may be acquired. For example, the digital scan may be done using specialized dental scanners that capture the precise shape and details of the oral cavity. Alternatively, traditional dental impressions can be taken using dental putty or alginate and then digitized through a process called scanning or molding.

In some embodiments, computer-aided design (CAD) software specifically designed for dental applications may be used to generate a usable 3D model based on the impression/digital scan. The CAD software allows for manipulation and modeling of the digital scan to create a customized bite guard design.

In some embodiments, the system may receive one or more parameters of the bite guard. For example, such parameters may include structural elements or features to enhance the bite guard's strength and performance, the thickness of the material, the coverage area, the alignment with teeth and gums, and any additional features required for protection or comfort.

In some embodiments, the system may test and validate the design prior to 3D printing. In some embodiments, the system simulates the use of the bite guard design using CAD software or specialized dental software tools. In some embodiments, simulation allows for virtual testing and analysis to ensure the bite guard meets the necessary criteria, such as fit, comfort, protective capabilities, and chip distribution.

In some embodiments, once the design has been validated, operation 410 can export the finalized 3D bite guard model as a suitable file format, such as STL (Standard Tessellation Language or Standard Triangle Language) or another format used in 3D printing.

In some embodiments, the system may use slicing software, dictated by the chosen 3D printing technology, to make a printing file. In some instances, the slicing software may configure parameters such as layer thickness, infill density, and support structures to optimize the printing process and ensure a successful print.

Method 400 continues with operation 420 of fabricating a bite guard.

Fabricating a bite guard involves the utilization of various methods, including 3D printing, molding, vacuum forming and other techniques. In some embodiments, 3D printing offers precise customization and rapid production of bite guards. The 3D printing process allows for intricate details and personalized features, such as individualized fit and varying thicknesses in different areas of the bite guard for enhanced protection.

In some embodiments, once the CAD file is ready, it is sent to a 3D printer for fabrication. The 3D printing process utilizes additive manufacturing technology, which builds the bite guard layer by layer. The printer translates the digital design into physical layers by depositing material in a controlled manner.

In some embodiments, the 3D printer uses a filament material with embedded materials for printing the bite guard (e.g., a compliant biocompatible polymer). The filament is loaded into the printer. The printer's extruder or printhead then melts or liquefies the material and deposits it onto the build plate or previous layers according to the instructions provided in the CAD file.

In some embodiments, the printer precisely controls the deposition of the material, following the contours and features defined in the digital design. As each layer is completed, it solidifies and fuses with the previous layers, forming a cohesive structure.

In some embodiments, once the printing process is finished, the bite guard is carefully removed from the printer. Depending on the printing technology used, additional post-processing steps may be required. These can include removing support structures if they were used during printing, cleaning, or smoothing the surface of the bite guard, and performing any necessary quality checks or inspections.

In some instances, after post-processing, the bite guard may undergo further adjustments or modifications to ensure a proper fit. This can involve trimming or shaping specific areas to match the individual's dental anatomy or comfort preferences.

In some embodiments, the final result is a customized, 3D printed bite guard that is tailored to the specific requirements of the wearer. The advantages of 3D printing in bite guard fabrication include precise customization, rapid production, and the ability to incorporate intricate features or variations in thickness for optimal protection and comfort.

In some embodiments, the mouth guard is placed in the patient's mouth and the array of piezo sensor assemblies is connected to an external device. In some embodiments, wirelessly connecting an array of piezo sensor assemblies to an external device includes establishing a communication link that enables seamless data transfer between the sensors and the device. This is typically achieved using wireless communication technologies such as Bluetooth®, Wi-Fi, or Zigbee®. The external device, such as a smartphone, tablet, or computer, is equipped with a corresponding wireless receiver that can receive and interpret the sensor data. In some instances, wireless connectivity eliminates the need for physical wiring and provides flexibility in sensor placement and data acquisition. In some embodiments, wirelessly connecting to the array of piezo sensor assemblies in the bite guard enables real-time monitoring, data analysis, and control of the sensors from a central device, opening up possibilities for diverse applications in fields like healthcare, environmental monitoring, and industrial automation. In some embodiments, the array of piezo sensor assemblies includes multiple piezo sensor assemblies, as described herein, randomly distributed throughout regions of a bite guard.

Method 400 continues with operation 430 of receiving pressure data from the chips in the bite guard. In some embodiments, the pressure data includes an orientation of the chip, including the orientation of the pressure sensor. As described herein, in some embodiments, The bite guards are made from biocompatible, soft filaments that allow the pressure from the teeth to be accurately transferred to the embedded piezo sensor assembly.

Method 400 continues with operation 440 of generating a time-dependent pressure map of the pressure generated by a patient's teeth. In some embodiments, the pressure map may be made on a handheld device with components similar to computer system 201.

In some embodiments, the time dependent pressure map includes a 3D force vector map. In some instances, A pressure map is a graphical representation that illustrates the distribution of pressure across a surface or object. It provides a visual depiction of how pressure varies across different regions, indicating areas of high or low pressure. Pressure maps are commonly used in various fields such as medicine (for assessing contact pressure on body parts), engineering (to analyze stress distribution on structures), and ergonomics (for evaluating pressure points on seating or surfaces). By visualizing pressure patterns, pressure maps help identify potential areas of discomfort, stress, or imbalance.

In some instances, a 3D force vector map represents the magnitude and direction of forces acting on a three-dimensional object or within a three-dimensional space. It provides a comprehensive view of how forces are distributed and oriented on the chips. The map consists of arrows or vectors, where the length represents the magnitude of the force, and the direction indicates its orientation. By visualizing the force vectors, it becomes possible to assess the overall force distribution, identify areas of high or low force, and analyze the balance and stability of the system.

In some embodiments, the time-dependent pressure map may aid the diagnosis and treatment of teeth grinding problems much more effective than using traditional methods, and the bite guard itself is patient-specific and disposable, making it a cost-effective and efficient invention solution.

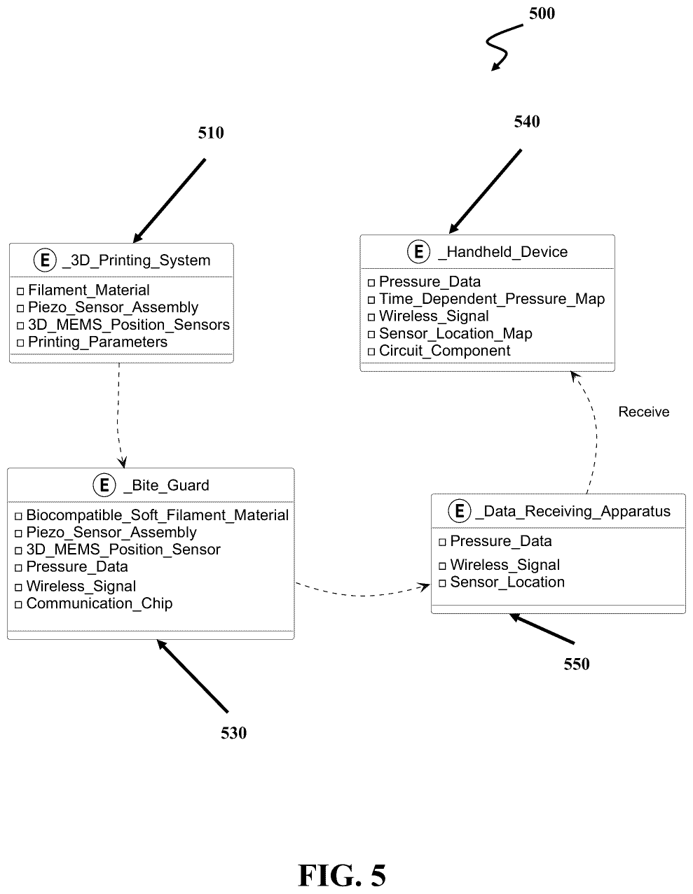

FIG. 5 depicts an Entity Relationship management Diagram (ERD) for an example bite guard system 500 as described herein. Each entity is labeled with an E for entity. Although 3D printing system 510 is depicted, other methods of fabricating a bite guard with Piezo sensors and position sensors may be used with other embodiments herein. For example, a bite guard could be molded with resin where sensors have been suspended in the resin. In some embodiments, 3D printing system 510 may use a filament material with piezo sensors and 3D MEMS position sensors to fabricate bite guard 530.

In some instances, an Entity Relationship management Diagram (ERD) is a visual representation of data relationships between entities. It illustrates how entities may relate to one another, how they store and retrieve data, and how it can communicate with other entities. ERDs are typically used in database design and computer programming to represent and analyze data. An ERD outlines the structure of a database with descriptors such as entities, attributes, relationships, and keys. The ERD also shows how data is to be stored and retrieved by the database. Its purpose is to visually represent the relationship between entities in the database and ensure that the database design properly follows business rules and regulations.

In some embodiments, bite guard 530 may be composed of a biocompatible soft material with embedded piezo sensor assemblies (formed during printing from biocompatible soft filament material with embedded piezo sensor assemblies). In some embodiments, the piezo sensor assemblies (sensor assemblies herein) may also include 3D MEMS position sensors and communications chips (capable of sending a wireless signal with pressure data, location data, and orientation data).

In some embodiments, the wireless signal may be received by data receiving apparatus 550 connected to integrated in handheld device 540. In some embodiments, the data receiving apparatus 550 may receive the wireless signal with pressure data for each piezo sensor assembly, location data for each piezo sensor assembly, and orientation data for each piezo sensor assembly from the communication chips.

In some embodiments, handheld device 540 may use the pressure data for each piezo sensor assembly, location data for each piezo sensor assembly, and orientation data for each piezo sensor assembly to generate a time dependent pressure map for a patient's teeth and a sensor location map for the array so piezo sensor assemblies in the bite guard.

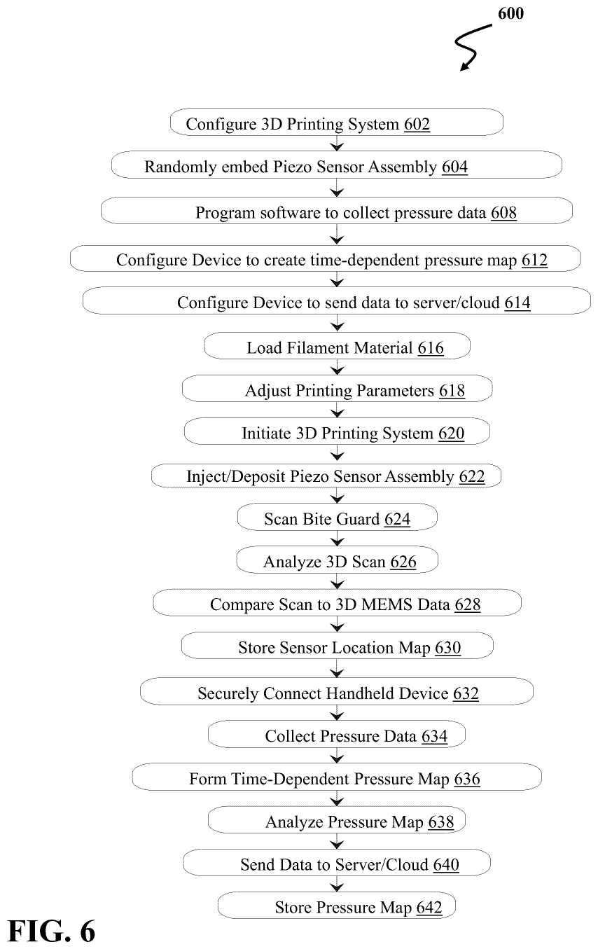

FIG. 6 depicts an example method 600 of fabricating a bite guard, measuring pressure and orientation data from embedded sensor assemblies, and analyzing the data to form a pressure map. In some embodiments, operations in method 600 may be enacted by code 200.

FIG. 6 begins with operation 602 of configuring a 3D printing system. In some embodiments, a system may configure the 3D printing system to use the biocompatible, soft filament material, such as Polylactic Acid (PLA), Acrylonitrile-Butadiene-Styrene (ABS), Polyethylene Terephthalate (PETG), and Thermoplastic Elastomer (TPE), that can transfer the pressure from the teeth to the embedded piezo sensor assembly without buffering or distorting the pressure map.

FIG. 6 continues with operation 604 of randomly embedding piezo sensor assemblies in filament material. In some embodiments, the piezo sensor assemblies may include 3D MEMS position sensors, and communications chips. In some embodiments, the 3D MEMS position sensors may be included in the piezo sensor assemblies to determine the orientation of the randomly positioned piezo sensor. In some instances, the 3D MEMS position sensors use accelerometers and gyroscopes to detect the orientation of the sensor relative to the teeth surface, allowing for accurate calculation of the force vectors.

The piezo sensor assemblies (such as example piezo sensor assemblies 320) may be released into the solution and may then be functionalized. The piezo sensor assemblies 320 (e.g., sensor chips) can be dispersed in a polymer carrier in an aqueous solution or organic solvent.

In some embodiments, for organic solvent dispersion, the piezo sensor assemblies 320 can be first coated with monolayers of alkylsilanes by immersing the piezo sensor assemblies 320 in a dilute (e.g., 0.1-1%) solution of alkyl trimethoxysilane and then rinsing with the solvent (e.g., ethanol, water, or a mixture thereof). In this process, the surface of the piezo sensor assemblies can be coated with monolayers of long chain (2-16 carbon atoms) alkyl group which may allow for dispersal in a carrier and prevent agglomeration of the piezo sensor assemblies 320. Coated piezo sensor assemblies 320 can be added to a solution of a polymer such as, for example, nitrocellulose (2-10% by solid) in ethyl or butyl acetate and stirred or sonicated to form a uniform dispersion.

In some embodiments, for aqueous dispersion of the piezo sensor assemblies 320, the piezo sensor assemblies 320 can be first coated, for example, with polyetheneoxide endcaped with trialkoxysilane to form a hydrophilic monolayer on the piezo sensor assemblies 320. The coated piezo sensor assemblies 320 are then dispersed in an aqueous solution, for example, of a mixture of polyvinylpyrrolidone and polyethyleneoxide diacrylate.

In some embodiments, the above steps may be incorporated in a solution blending process so that the piezo sensor assemblies 320 are dispersed in a polymer blend. Polymer blends are widely used for 3D and 4D printing. A polymer blend may refer to a blended mixture of two or more polymers. A polymer blend may also refer to a blended mixture of one or more polymers with other materials such as ceramics, carbon nanostructures or other fillers. The polymers may include, among other things, polylactic acid (PLA), acrylonitrile butadiene (ABS), polyethylene terephthalate glycol (PETG), polypropylene (PP), carbon fiber, nylon, high-impact polystyrene (HIPS), thermoplastic elastomers, or any other suitable polymer.

Polymer powder with embedded piezo sensor assemblies 320 may be produced from the solution. For example, the solution may be cooled down from 80-100° C. to room-temperature to induce precipitation of polymer particles comprised of polymer grains with embedded piezo sensor assemblies 320, separating the precipitate, drying, and mechanical treatment (milling, grinding, chipping, etc.).

In some embodiments, the plastic (i.e., polymer) powder with embedded piezo sensor assemblies 320 may be used to produce plastic pellets with the embedded piezo sensor assemblies 320. Each plastic grain in the powder or a plastic pellet may contain one or more (e.g., thousands or more) piezo sensor assemblies 320, which may be incorporated into the filament and then the 3D/4D printed structure. The plastic pellets with embedded piezo sensor assemblies 320 may be used to produce filaments with embedded piezo sensor assemblies 320. Filaments may also be produced directly from powder. It may be appreciated that substantially any technique, such as melt-blending, used for producing filaments from powders or pellets may be used. The filaments with embedded piezo sensor assemblies 320 can be used for printing 3D or 4D structures with embedded sensors. It may be appreciated that no change is needed in the 3D or 4D printing step because of the embedded sensors (i.e., piezo sensor assemblies 320).

FIG. 6 continues with operation 608 of programing software to collect pressure data. In some embodiments, to wirelessly connect an array of piezo sensor assemblies, such as a piezo sensor assembly, to an external device for data collection and processing, several technical steps are involved. Firstly, the sensors need to be interfaced with a microcontroller or a similar embedded system that can read the sensor outputs. The microcontroller is responsible for collecting the pressure data from the piezo sensors.

In some embodiments, the software running on the microcontroller needs to be programmed to acquire the sensor data. This involves configuring the microcontroller's input/output pins to interface with the piezo sensors and utilizing appropriate analog-to-digital conversion techniques to convert the analog pressure signals into digital form.

In some embodiments, once the sensor data is collected and converted, the microcontroller needs to establish a wireless communication link with the external device, such as a handheld device. This can be achieved by integrating a wireless module, such as a Bluetooth® or Wi-Fi module, into the microcontroller. The module enables wireless transmission of the sensor data to the handheld device.

In some embodiments, in a device, a compatible wireless receiver module is required to receive the transmitted data. The receiver module could be integrated into the device or connected externally via a USB or other interface. The handheld device may also have software or an application installed that can receive and process the sensor data.

The software running on the handheld device needs to be programmed to establish a connection with the microcontroller and receive the transmitted sensor data. This involves implementing the necessary protocols and communication interfaces to establish a reliable wireless link between the devices.

Once the sensor data is received on the handheld device, the software can process and analyze the data as per the desired application. This could involve performing calculations, generating visualizations, or triggering certain actions based on specific thresholds or patterns in the pressure data.

FIG. 6 continues with operation 612 of configuring the device to create time-dependent pressure maps.

To create a time-dependent pressure map, an advanced data acquisition system is used to collect pressure data from multiple sensors at regular intervals. The pressure sensors, strategically placed in specific locations, capture real-time pressure readings. These sensors are connected to a data acquisition module capable of recording and time-stamping the pressure data with high precision. The time interval for data sampling is carefully chosen based on the requirements of the application, considering the dynamics of the system and the desired temporal resolution of the pressure map.

The data acquisition module continuously captures pressure readings from each sensor at the defined time interval throughout the desired duration of the measurement. The collected data is then stored in a database or saved in a suitable file format for further analysis. To process the data, specialized software or programming tools are employed. The recorded pressure data is processed using algorithms and statistical techniques to derive meaningful insights.

Visualization techniques play a useful role in presenting the time-dependent pressure map. Sophisticated software tools generate visual representations of the data, such as heatmaps, contour plots, or animations, which enable a comprehensive analysis of pressure variations over time. These visualizations allow for the identification of trends, patterns, and anomalies in the pressure distribution, contributing to a deeper understanding of the system under investigation.

The time-dependent pressure map serves a range of technical applications, including fluid dynamics studies, mechanical system analysis, and real-time pressure monitoring in various industries. By accurately capturing and visualizing pressure changes over time, this approach provides valuable insights into the behavior and performance of systems, enabling informed decision-making and optimization of processes.

FIG. 6 continues with operation 614 of configuring the device to send data to a server/cloud.

To configure the device for wireless data transmission, several technical considerations need to be addressed. First, the device must be equipped with wireless communication capabilities, such as Wi-Fi or cellular connectivity. This can be achieved through the integration of wireless modules or utilizing built-in wireless capabilities of the device.

Once the wireless communication is established, the device needs to be configured to establish a connection with a server or a cloud platform. This involves setting up the appropriate network protocols and security measures to ensure secure and reliable data transmission. Depending on the specific requirements, industry-standard communication protocols such as HTTP, MQTT, or WebSocket can be utilized.

To enable seamless data transfer, the device's software must be programmed to package the pressure data into a suitable format for transmission. This typically involves converting the data into a structured format, such as JSON or XML, which facilitates easy parsing and processing on the server or cloud side. Additionally, data compression techniques may be employed to minimize the size of the transmitted data, optimizing bandwidth utilization.

In some embodiments, authentication and encryption mechanisms may be implemented to ensure data security during transmission. This involves establishing secure communication channels using protocols like TLS (Transport Layer Security) or SSL (Secure Sockets Layer), which encrypt the data to prevent unauthorized access or tampering.

In some embodiments, the server or cloud platform must be properly configured to receive and process the incoming pressure data. This includes setting up the necessary storage infrastructure to store the received data and implementing data processing algorithms or analytics tools to analyze the data in real-time or offline. The server may also be integrated with other systems or databases for seamless data integration and interoperability.

In some embodiments, once the pressure data is received and stored on the server or cloud, it can be accessed and analyzed by healthcare providers or authorized users. Visualization tools, data analytics algorithms, and machine learning techniques can be applied to gain insights from the data and support decision-making processes. The data can also be securely shared with healthcare providers or other stakeholders using appropriate access controls and privacy measures, ensuring compliance with data protection regulations.

FIG. 6 continues with operation 616 of loading filament material into the 3D printer.

In some embodiments, the filament material of choice is loaded into the 3D printing system. Common options for filament materials include PLA (polylactic acid), ABS (acrylonitrile butadiene styrene), PETG (polyethylene terephthalate glycol), or TPE (thermoplastic elastomer). The selection of filament material depends on factors such as the desired softness, flexibility, and biocompatibility required for the final product.

In some embodiments, PLA filament may be used for its ease of use, biodegradability, and low toxicity. Advantageously, PLA provides relatively rigid yet comfortable properties. PLA provides a good balance between strength and flexibility, making it suitable for protecting the teeth and gums during physical activities.

In some embodiments, ABS filament may be used when higher impact resistance and durability are required. ABS is known for its toughness, making it suitable for bite guards that may be subjected to greater forces or harsher conditions.

In some embodiments, PETG filament is used for its enhanced flexibility and transparency. PETG offers excellent impact resistance and durability while maintaining a softer feel compared to PLA or ABS. This makes it suitable for bite guards that require a balance between protection and comfort, allowing for slight flexibility to accommodate the wearer's bite.

In some embodiments, TPE filament can be chosen for its exceptional elasticity and softness. TPE materials exhibit rubber-like properties, providing a high level of comfort and flexibility. TPE-based bite guards offer excellent shock absorption and can conform well to the contours of the wearer's teeth and gums.

FIG. 6 continues with operation 618 of adjusting printer parameters.

In some embodiments, the printing parameters of the 3D printing system are adjusted to achieve the desired characteristics of the bite guard. These parameters include temperature, speed, and layer height, which directly affect the final outcome of the printed object.

In some embodiments, the temperature of the 3D printing system is carefully controlled to ensure optimal material flow and bonding. For soft bite guards, a lower printing temperature may be used to maintain the desired flexibility and softness of the material. This allows the bite guard to effectively absorb and transfer the pressure from the teeth to the embedded piezo sensor assembly.

In some embodiments, the printing speed is adjusted to strike a balance between accuracy and efficiency. A slower printing speed allows for better precision in capturing the intricate details of the bite guard design, ensuring that the embedded piezo sensor assembly is properly encapsulated. This helps to maintain the integrity and functionality of the sensor system.

In some embodiments, the layer height, or the thickness of each printed layer, is carefully chosen to achieve the desired level of detail and comfort. A smaller layer height results in finer resolution and smoother surface finish, contributing to a more comfortable fit of the bite guard. It allows for better integration of the piezo sensor assembly, ensuring that the pressure from the teeth is accurately transferred to the sensors.

By adjusting these printing parameters, the 3D printing process can create a bite guard that meets the required softness while accurately transferring the pressure from the teeth to the embedded piezo sensor assembly. It is beneficial to refine these parameters to ensure the functionality, comfort, and performance of the bite guard as a reliable sensing device for pressure analysis.

FIG. 6 continues with operation 620 of initiating the 3D printing system.

In some embodiments, the 3D printing process is initiated by initializing the 3D printing system. The system is set up with the necessary parameters and materials for printing the bite guard. A predetermined pattern, typically designed using computer-aided design (CAD) software, is loaded into the 3D printing system. This pattern serves as a blueprint for the precise construction of the bite guard.

The printing process begins as the 3D printer follows the predetermined pattern layer by layer. The printer's extrusion mechanism deposits the chosen filament material, such as PLA, ABS, PETG, or TPE, in a controlled manner. The material is heated to its melting point and extruded through a nozzle, forming each layer of the bite guard.

As each layer is completed, the build platform gradually moves down or the print head moves up, depending on the printer's design, allowing for the construction of subsequent layers. This layer-by-layer approach ensures the accurate replication of the predetermined pattern, resulting in a bite guard with the desired shape, dimensions, and features.

In some embodiments, throughout the printing process, the 3D printer's software controls various aspects, including the movement of the print head, temperature regulation, filament flow, and overall printing parameters. This ensures precise execution of the predetermined pattern, maintaining consistency and quality throughout the fabrication of the bite guard.

In some instances, once the printing process is completed, the bite guard undergoes a cooling period to solidify and stabilize its structure. After cooling, the printed bite guard is ready for post-processing steps, which may include trimming excess material, smoothing rough edges, and/or applying any necessary finishing touches to achieve the desired aesthetics and functionality.

FIG. 6 continues with operation 622 of injecting/depositing the filament with the randomly embedded piezo sensor assemblies into the form of a mouthguard. In some embodiments, as the 3D printing system is printing the bite guard, the randomly embedded piezo sensor assembly is injected or directly deposited onto the filament material in a predetermined pattern. For example, as described above, the filament material may already have piezo sensor assemblies embedded into it. In some embodiments, the piezo sensor assemblies may be deposited into the filament during printing. For example, the assemblies may be inserted into the resin before it cools, sets, or cures. For example, the assemblies may be placed on the surface of a completed layer before the next layer is printed.

In some embodiments, the system fabricates a 3D-printed structure having one or more embedded piezo sensor assemblies. The 3D printed structure may include successively formed layers of filament One or more of the layers of filament may include regions of filament having embedded piezo sensor assemblies. By registering the sensors and their location within the structure, the locations of the regions, therefore, may be known to a user of the 3D-printed structure.

In some embodiments, some regions that require pressure measurements may be fabricated using filament with embedded piezo sensor assemblies and other regions may be printed with a similar filament not containing embedded piezo sensor assemblies. For example, the sides of the bite guard may not need to have pressure sensors since the sides do not have substantial force exerted on them by a patient's teeth.

FIG. 6 continues with operation 624 of scanning the fabricated bite guard in 3D. In some embodiments, the bite guard is scanned to create a sensor location map, which can be used to identify the location of each of the embedded piezo sensors. In some embodiments, the bite guard is placed on the scanning platform. The scanning platform is a flat, level surface that leaves enough room for the bite guard to fit comfortably.

In some embodiments, the scanning software is initiated, and the bite guard is scanned using a 3D scanner to create a 3D model of the bite guard with the embedded piezo sensor assemblies. The 3D scanner uses lasers and cameras to capture a high-resolution image of the bite guard and the embedded sensors.

In some embodiments, the process of scanning an object for MEMS sensor locations involves a systematic procedure using specialized equipment and techniques. The objective is to identify specific areas or regions on the object where MEMS sensors will be positioned or embedded.

In some embodiments, prior to scanning, the object and the requirements for MEMS sensor placement are assessed. In some instances, assessment includes understanding the desired sensor positions, orientations, and any constraints imposed by the object's shape or material. The necessary scanning equipment, such as a 3D scanner or coordinate measuring machine (CMM), is set up and calibrated.

In some embodiments, depending on the size and complexity of the object, different scanning methods may be employed. One approach is using a 3D scanner that utilizes techniques like laser scanning, structured light projection, or photogrammetry. The scanner captures the object's surface geometry and texture data, providing a digital representation of its shape.

In some embodiments, the object is placed on a stable surface, and the scanning process is initiated. For example, the scanner emits laser beams, projects structured light patterns, or captures multiple images from different angles to gather data about the object's surface. The scanning equipment precisely measures the object's dimensions, capturing both its external features and internal details.

In some embodiments, as the scanning progresses, the equipment collects data points that represent the object's surface topology. The acquired data is typically stored in a point cloud format or a 3D mesh representation, which contains information about the object's geometry, texture, and color.

In some embodiments, once the scanning is complete, the acquired data is processed to extract relevant information about the object's surface and identify suitable locations for MEMS sensor placement. For example, the processing may involve analyzing the point cloud or mesh data, applying algorithms to detect specific features or regions, and segmenting the object's surface based on predefined criteria.

In some embodiments, by overlaying the scanned data with the predefined sensor placement requirements, the scanned object's surface is mapped to identify locations for embedded MEMS sensors.

In some embodiments, the final output of the scanning process is a detailed map or reference guide indicating the specific locations on the object where MEMS sensors are positioned. This information can be further verified and refined through visual inspection, physical measurements, or additional analysis techniques to ensure accurate sensor location.

FIG. 6 continues with operation 626 of analyzing the 3D scan of the bite guard.

In some embodiments, the 3D scan is then analyzed by the software to create a sensor location map, which identifies the position of each of the embedded sensors. The software uses algorithms to determine the location of each sensor in the bite guard, relative to the other sensors.

In some embodiments, the sensor location map is then compared to the 3D MEMS position sensors data to determine the orientation of each sensor relative to the teeth surface. The 3D MEMS data indicates the exact orientation of each sensor and allow for accurate calculation of the force vectors.

In some embodiments, analyzing the scan of the object involves a comprehensive examination of the acquired data to extract meaningful insights and valuable information about its surface characteristics.

In some embodiments, the scanned data is imported into specialized software or processing tools designed for point cloud or mesh data analysis. This software enables various analytical operations and algorithms to be applied to the scanned data for further evaluation.

In some embodiments, one of the initial steps in the analysis process is data filtering and noise reduction. This involves removing any outliers or irregularities in the scanned data that may have occurred during the scanning process or are artifacts of the surface texture. Noise reduction techniques, such as statistical filters or outlier removal algorithms, are employed to enhance the quality and accuracy of the data.

In some embodiments, surface reconstruction algorithms are applied to the scanned data to create a continuous and smooth representation of the object's surface. These algorithms aim to interpolate the point cloud or mesh data to generate a coherent surface model that accurately reflects the object's shape and geometry.

In some embodiments, after surface reconstruction, geometric features extraction techniques are utilized to identify specific surface characteristics of interest. These techniques involve detecting edges, corners, or other distinctive geometric elements that can provide valuable information about the object's structure or design.

In some embodiments, advanced analysis techniques such as surface deviation analysis or curvature analysis may be employed to quantify and visualize deviations or variations from the object's ideal surface. These analyses help assess the object's dimensional accuracy, identify areas of concern, or evaluate the quality of its surface finish.

In some embodiments, the scanned data can also be compared or aligned with a reference model or CAD (Computer-Aided Design) data to assess the object's conformance to the intended design. This alignment process enables deviation analysis, geometric comparisons, and measurements to be performed, providing valuable feedback on the object's manufacturing accuracy.

In some embodiments, additional analysis tasks may involve color mapping or texture analysis to extract surface texture information from the scanned data. This can be useful for applications where visual appearance or texture properties of the object are of significance.

In some embodiments, the results of the analysis process are presented through visualizations, reports, or measurements that highlight the relevant characteristics of the scanned object.

FIG. 6 continues with operation 628 of comparing the scan to 3D MEMS data. In some embodiments, the scan may give the placement of one or more sensor assemblies in the array. In some embodiments, the positional data from the 3D MEMS sensor may be used in conjunction with the scan to determine exact placement and orientation of each sensor assembly in the array.

FIG. 6 continues with operation 630 of storing the sensor location map on a cloud/server.

In some embodiments, storing a file on a cloud or server involves compressing and preparing the for storage. A secure connection is established between the client device and the storage service. The sensor location map is then transmitted securely to the cloud or server. The storage service allocates space for the sensor location map and implements redundancy measures for data integrity. Additional protection measures, such as encryption at rest, may be applied. Sensor location map management features, backups, and data synchronization ensure durability and availability. Logging and auditing mechanisms track sensor location map access and modifications for security and compliance purposes.

FIG. 6 continues with operation 632 of securely connecting a handheld device.

In some embodiments, the handheld device is connected to the system via a USB or Bluetooth® connection. This connection allows the device to receive the pressure data from each of the piezo sensors embedded in the bite guard.

FIG. 6 continues with operation 634 of collecting pressure data. In some embodiments, the pressure data from the piezo sensor assembly is collected by the device and processed. The pressure data is collected over a period of time and stored in the device's memory.

FIG. 6 continues with operation 636 of generating a time-dependent pressure map from the pressure data and the sensor location map. In some embodiments, the pressure data is then used to create a time-dependent pressure map of the person's teeth. The pressure data is analyzed and processed to create a map that shows the pressure being applied to each tooth over a period of time.

FIG. 6 continues with operation 638 of analyzing the pressure map. In some embodiments, the pressure map is then analyzed by a healthcare provider to diagnose the root cause of the teeth grinding, and to provide a treatment plan to help stop the grinding. In some instances, the pressure map can be used to identify any areas of the teeth that are being subjected to excessive force and to pinpoint the source of the grinding.

FIG. 6 continues with operation 640 of sending the data to a server/cloud. In some embodiments, the data from the pressure map can also be sent to a server/cloud for further analysis or to be shared with a healthcare provider. This allows for remote monitoring of the patient's teeth grinding and allows for more accurate diagnosis and treatment.

FIG. 6 continues with operation 642 of storing the pressure map. In some embodiments, the pressure map is stored in the device's memory for later analysis. This allows the data to be accessed and reviewed at any time and allows the healthcare provider to track the progress of the treatment plan.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present disclosure have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to explain the principles of the embodiments, the practical application or technical improvement over technologies found in the marketplace, or to enable others of ordinary skill in the art to understand the embodiments disclosed herein.

What is claimed is:

1. A device comprising:
a bite guard configured to conform to teeth of a patient; and
an array of sensor assemblies encapsulated in the bite guard,
wherein the array of sensor assemblies is distributed throughout the bite guard; and
wherein each sensor assembly includes a piezo pressure sensor.

2. The device of claim 1,
wherein the bite guard is 3D printed.

3. The device of claim 2, wherein the array of sensor assemblies is embedded in a printing material prior to printing.

4. The device of claim 3, wherein a density of the array of sensor assemblies in the printed bite guard is based on the density of sensor assemblies in a filament used for the printing.

5. The device of claim 2, wherein the bite guard is printed with a first material containing an array of embedded sensor assemblies, and a second material that does not contain any embedded sensor assemblies.

6. The device of claim 1, wherein the bite guard is made from a biocompatible, soft filament material.

7. The device of claim 1, wherein each sensor assembly in the array of sensor assemblies comprises a communication chip and a 3D orientation MEMS chip.

8. The device of claim 1, wherein an orientation of the piezo pressure sensor is random.

9. A system comprising:
a memory storing program instructions; and
a processor in communication with the memory, the processor being configured to execute the program instructions to perform processes comprising:
detecting, by an array of piezo sensor assemblies in a bite guard, pressure measurements applied by a patient to the bite guard; and
transmitting, from the array of piezo sensor assemblies in the bite guard to the processor, the pressure measurements.

10. The system of claim 9, wherein the memory stores further program instructions, and wherein the processor is configured to execute the further program instructions to perform the processes further comprising:
detecting, by the array of piezo sensor assemblies, location data for the array of piezo sensor assemblies; and
transmitting, from the array of piezo sensor assemblies in the bite guard to the processor, the location data.

11. The system of claim 10, wherein the memory stores further program instructions, and wherein the processor is configured to execute the further program instructions to perform the processes further comprising:
mapping locations of the array of piezo sensor assemblies in the bite guard.

12. The system of claim 11, wherein the memory stores further program instructions, and wherein the processor is configured to execute the further program instructions to perform the processes further comprising:
generating, based on the mapping of the locations and the pressure measurements, a time-dependent pressure map.

13. The system of claim 9, wherein the bite guard is fabricated by 3D printing with a filament that contains multiple embedded piezo sensor assemblies.

14. The system of claim 9, wherein each embedded piezo sensor assembly contains a piezo pressure sensor, a MEMS (Micro-Electro-Mechanical Systems) position sensor, and a communication chip.

15. A computer program product comprising a computer readable storage medium having program instructions embodied therewith, the program instructions executable by a processor to cause the processor to perform a method, the method comprising:
detecting, by an array of piezo sensor assemblies in a bite guard, pressure measurements applied by a patient to the bite guard; and
transmitting, from the array of piezo sensor assemblies in the bite guard to the processor, the pressure measurements.

16. The computer program product of claim 15, further comprising additional program instructions stored on the computer readable storage medium and configured to cause the processor to perform the method further comprising:
detecting, by the array of piezo sensor assemblies, location data for the array of piezo sensor assemblies; and
transmitting, from the array of piezo sensor assemblies in the bite guard to the processor, the location data.

17. The computer program product of claim 16, further comprising additional program instructions stored on the computer readable storage medium and configured to cause the processor to perform the method further comprising:
mapping the array of piezo sensor assemblies with an individual location and orientation of the sensors in the bite guard.

18. The computer program product of claim 17, further comprising additional program instructions stored on the computer readable storage medium and configured to cause the processor to perform the method further comprising:
generating, based on the mapping of the location and the pressure measurements, a time-dependent pressure map.

19. The computer program product of claim 18, wherein the location data includes location and orientation data for one or more sensors in the array of piezo sensor assemblies.

20. The computer program product of claim 15, wherein each sensor assembly, in the array of piezo sensor assemblies includes a piezo pressure sensor, a MEMS (Micro-Electro-Mechanical Systems) position sensor, and a communication chip.
